(19) United States
(12) Patent Application Publication
(10) Pub. No.: US 2010/0233130 A1
(43) Pub. Date: Sep. 16, 2010

(54) Method and Apparatus for Maintenance and Expansion of Hematopoietic Stem Cells From Mononuclear Cells

(75) Inventor: Shai Meretzki, Haifa (IL)

Correspondence Address: MARTIN D. MOYNIHAN d/b/a PRTSI, INC., P.O. BOX 16446, ARLINGTON, VA, 22215, US

(73) Assignee: Pluristem Ltd., Haifa (IL)

(21) Appl. No.: 12/223,634

(22) PCT Filed: Feb. 6, 2007
(86) PCT No.: PCT/IL2007/000157

§ 371 Date: Aug. 5, 2008

Related U.S. Application Data
(60) Provisional application No. 60/765,218, filed on Feb. 6, 2006.

(51) Int. Cl. C12N 5/077; A61K 35/12

(52) U.S. Cl. 424/93.7; 435/373

(57) ABSTRACT

A method of expanding/maintaining undifferentiated hematopoietic stem cells by obtaining unselected mononuclear cells; and seeding the mononuclear cells into a stationary phase plug-flow bioreactor in which a three dimensional mesenchymal/stromal cell culture has been pre-established, thereby expanding/maintaining undifferentiated hematopoietic stem cells.

### FIELD AND BACKGROUND OF THE INVENTION

The present invention relates to a method and apparatus for maintenance and expansion of hematopoietic stem cells using non-selected mononuclear cells. More particularly, the present invention relates to the maintenance and/or expansion of hematopoietic stem cells from unselected mononuclear cells for the maintenance and/or expansion of such hematopoietic stem cells.

The hematopoietic system in mammals is composed of a heterogeneous population of cells that range in function from mature cells with limited proliferative potential to pluripotent stem cells with extensive proliferative, differentiative and self renewal capacities (1-3). Hematopoietic stem cells (HSC) are exclusively required for hematopoietic reconstitution following transplantation and serve as a primary target for gene therapy. In spite of the key role of stem cells in maintaining the hematopoietic system, there are significant obstacles to therapeutic applications: hematopoietic stem cells are found in extremely low proportions in hematopoietic tissue. Methods for growth and expansion of undifferentiated stem cells under ex-vivo conditions for prolonged periods have meet with limited success.

It is widely accepted that stem cells are intimately associated in vivo with discrete niches within the marrow (4-6), which provide molecular signals that collectively mediate their differentiation and self renewal, via cell-cell contacts or short-range interactions (7) and the production of growth factors. These niches are part of the “hematopoietic inductive microenvironment” (HIM), composed of marrow stromal cells, e.g., macrophages, fibroblasts, adipocytes and endothelial cells (8). Marrow stromal cells maintain the functional integrity of the HIM by providing extracellular matrix (ECM) proteins and basement membrane components that facilitate cell-cell contact (9-11). They also provide various soluble or resident cytokines needed for controlled hematopoietic cell differentiation and proliferation (12-14).

SCID Repopulating Cells (SRC) are defined as hematopoietic stem cells which have the ability to home into the bone marrow of non-obese diabetic (NOD)/SCID mice (27), where it gives rise to human myeloid, lymphoid and erythroid cells and to early CD34+ progenitor populations (28-30). The repopulating cell fraction is exclusively found in hematopoietic cell fractions expressing the CD34+ surface antigen and lack the expression of CD38 (31) and its frequency in cord blood (1 per 3×10⁵ cells) is enriched as compared to bone marrow (1 per 9×10⁵ cells) or mobilized peripheral blood (1 per 6×10⁶ cells) (32). Recent studies have shown that the repopulating cell fraction resides within a subpopulation of CD34+38− cells expressing CXCR4, a surface receptor for the chemokine stromal cell-derived factor 1 (SDF-1, (34)) and apparently essential for homing and engraftment of human hematopoietic stem cells.

Recent attention has focused on the establishment of cytokine-supplemented suspension cultures for ex-vivo growth and expansion of hematopoietic stem cells, identifying important early-acting cytokines such as stem cell factor (SCF), FLT3 ligand and thrombopoietin (TPO). However, while being efficient in promoting proliferation, cytokine-assisted CD34+ expanded cells were demonstrated to have less engraftment potential than cytokine naive and unexpanded CD34+ cells (Xu & Reems 2001). Reconstitution of the marrow with this type of cultivated cells was found to be unsatisfactory in a variety of organisms ranging from mice to primates and human (Peters et al 1995; Peters et al 1996; Peters et al 2002; Glimm et al 2000; Drouet et al 2001; Cerny et al 2002; Mueller et al 2002; Ahmed et al 2004). Additionally, at least one of the cytokines being employed in hematopoietic stem cell expansion protocol—G-CSF—was shown to induce genetic and epigenetic alterations in the progenitor cells (Nagler et al 2004).

Studies aimed to induce prolonged maintenance/expansion of human hematopoietic stem cells on stromal cell monolayer cultures indicated failure to support the long-term maintenance and expansion of transplantable human hematopoietic stem cells on stromal cell layers. This may be due to the use of stromal cell monolayers, which do not reflect the in vivo growth conditions within the natural, three-dimensional structure of the bone marrow. Indeed, superior growth of a human hematopoietic cell line was observed using stromal cells seeded in a three dimensional collagen matrix, as compared to their proliferation on those cells monolayers. More importantly, a three dimensional tantalum-coated porous biomaterial, was shown to favorably enhance the short-term maintenance of Long Term Cultures Initiating Cells (LTCIC) or CD34+38− cells, as compared to cells cultured alone or on marrow stromal cell monolayers.

The capacity of stroma/mesenchymal cells to promote ex vivo expansion of undifferentiated hematopoietic stem cells when used in co-cultures as supporting cells has been demonstrated, and shown to be superior to monoculture expansion methods. While stroma/mesenchymal cells cultivated on flat, two dimensional surfaces or on spatially organized matrixes have been described (i.e. U.S. Pat. Nos. 5,541,107; 5,635,386; 5,674,750; 6,338,942; 6,642,049 and Rios and Williams 1990; Moore et al 1997; Majumdar et al 2000). However, none of these methods for ex vivo cultivation of hematopoietic stem cells have successfully replicated the marrow-like organization of the culture system, and all fail to promote expansion of hematopoietic stem cells while preventing their differentiation into more mature cells. Merchav et al. have recently disclosed a unique comprehensive hematopoietic stem cell ex-vivo expansion system that mimics the bone marrow microenvironment by using a spatial scaffold populated with mesenchymal cells to produce large amounts of undifferentiated, expanded hematopoietic stem cells. U.S. Pat. No. 6,911,201 to Merchav et al discloses a method of growing and expanding undifferentiated transplantable hematopoietic cells by culturing selected populations of early hematopoietic cells in a stationary phase plug-flow bioreactor on a three dimensional stromal cell cultures. Expansion of CD34+CD38− cells grown in the bioreactors continued effectively even in long term (7-14 days) cultures, and was superior to similar cells growth on stromal monolayers or unpopulated three dimensional scaffolds.

U.S. patent application Ser. Nos. 11/102,625, 11/102,654, 11/102,623 and 11/102,625, to Merchav et al, further disclose that conditioned medium from the three dimensional stromal culture as taught in U.S. Pat. No. 6,911,201 can effectively support the expansion and growth of hematopoietic stem cells in an undifferentiated state. Merchav et al. have demonstrated the effectiveness of their methods using pre selected population of CD34+ hematopoietic cells seeded on the stromal three dimensional culture, or grown in media containing stromal cell condition medium.

Hematopoietic and Mononuclear Cells:

Standard methodology for bone marrow reconstitution following induced myeloablation relies upon allogeneic hematopoietic stem cell transplantation. Presently; this risky clinical procedure has a mortality rate of 20-40%, for matched donors and an even higher mortality rate when the donor marrow is not from an HLA-identical sibling (Peters et al 1999).

The hematopoietic system in mammals is composed of heterogeneous population of cells that range in function from differentiated committed and mature cells with limited proliferative potential to pluripotent stem cells with extensive proliferative, differentiative and self renewal capacities (Turhan et al 1989; Morrison et al 1995; Gunsilius et al 2001; Bron et al 2002). Hematopoietic stem cells are the most primitive cells within the hematopoietic system. While partly differentiation-committed progenitors and differentiated cells make the vast majority of the hematopoietic cell population of any relevant source, the relative abundance of the true hematopoietic stem cells is very low. During hematopoietic reconstitution of the bone marrow in a myeloablated patient, differentiation committed progenitor cells are responsible for short-term hematopoietic recovery while the long-term hematopoiesis solely relies on the most primitive hematopoietic stem cells.

Many sources of stem cells may be involved in initiation of hematopoietic regeneration. The least differentiated of these cells are embryonic stem cells. These toti- to pluripotent cells possess the capacity to differentiate into any cell types. Likewise, embryonic stem cells could give rise in-vitro to form different blood cells (Willes and Keller 1991; Keller et al 1993). However, ethical and religious constrains limit their use. Also, the extremely primitive differentiative state of embryonic stem cells is associated with an inherent risk for teratoma formation (He et al 2002; Hovatta et al 2003; Wakitani et al 2003) and for imprinting-related developmental abnormalities (Humpherys et al 2001; Ogawa et al 2003). Accordingly, the use of embryonic stem cells is currently restricted to the realm of academic investigation.

Bone marrow is a preferred source of hematopoietic stem cells and transplantation of bone marrow-derived hematopoietic stem cells for marrow regeneration is a standard medical procedure. However, the use of bone marrow-derived hematopoietic stem cells is associated with several major drawbacks. The collection of bone-marrow aspirate is a surgical invasive procedure imposing medical threat on the donor, and there are also considerable risks on the recipient level, including viral transfection (Winston et al 1990; Schmidt et al 1991).

Since hematopoietic stem cells isolated from bone marrow-derived stem cells present obstacles for clinical use, peripheral blood and umbilical cord blood (CB) have recently been developed as alternative sources of hematopoietic stem cells. The major advantages of using these two sources include their availability and ease of collection. However, the abundance of hematopoietic stem cells in peripheral blood is the lowest of all accessible sources. On the other hand, cord blood transplantation is associated with durable engraftment and low incidence of severe graft-versus-host disease, even when 1-2 Human Leukocyte Antigens (HLA) mismatched cells are being employed (Rocha et al 2004). However, the major difficulty in using cord blood-derived hematopoietic stem cells for marrow recovery is their low absolute number in any given unit of cord blood, as clinical experience has established the importance of graft cell dose in determining the engraftment success and the patient's survival rate (Wagner et al 2002). When using cord blood derived hematopoietic stem cells for bone marrow reconstitution, high probability of survival is attained in recipients only when the graft contains 1.7×10⁵ CD34+ or more cells per kilogram of recipient's body weight. Since one unit of cord blood usually contains less than 5×10⁶ CD34+ cells, it allows for successful rescue of the bone marrow only in small weight individuals. Consequently, the limited ability to expand cord blood hematopoietic stem cells ex-vivo in a strict undifferentiated state remains a major obstacle to essential clinical applications, and developing efficient methods for hematopoietic stem cell expansion are important for the use of cord blood for effective bone marrow transplantation.

The challenge of ex vivo hematopoietic stem cell expansion originates from their predisposition to differentiate into more committed cells. Presently, hematopoietic stem cell expansion is accompanied by cellular differentiation, unless supported by feeder cells and/or signaling molecules. However, while efficiently proliferated ex-vivo, cytokine-assisted CD34+ expanded cells have inferior and unsatisfactory engraftment potential compared to cytokine naïve and unexpanded CD34+ cells (Xu & Reems 2001). Further, at least one of the cytokines used in hematopoietic stem cell expansion protocols—G-CSF was shown to induce genetic and epigenetic alterations in progenitor cells (Nagler et al 2004).

Apart from its supportive role in expansion of hematopoietic stem cells, stroma/mesenchymal cells have another advantage in that they inhibit T-cell proliferation and do not elicit immunological response to polyclonal stimuli. Practically, it has been shown that transplanting hematopoietic stem cells in combination with donor stroma/mesenchymal cells provides a very efficient engraftment process (Gurevitch et al 1999; Fan et al 2001; Almeida-Porada et al 2000).

Experimental and clinical hematopoietic stem cells ex vivo expansion methods usually employ CD34+ immuno-selected cells as the founding population. Indeed, it has been suggested that using supplemented cytokines, the immuno-selection phase is essential for the expansion process (Briddell et al 1997). However, CD34+ selection is associated with two substantial drawbacks. Firstly, CD34+ cells may not represent the earliest, most primitive hematopoietic stem cell type. Initially detected in mice, a CD34− cell subset which is able to reconstitute the bone marrow of a recipient has been identified (Osawa et al 1996; Morel et al 1998; Lange et al 1999) and it was demonstrated that this cohort of CD34− cells also contain the pool of precursors for the CD34+ cells subpopulation (Zanjani et al 1998; Ando et al 2000). It is hypothesized that the CD34⁻ stem cells hematopoietic stem cells are extremely quiescent and that some type of activation is required to cause up-regulation of CD34 expression and induce engraftment capacity. Secondly, great loss of target cell population is associated with presently employed immuno-selection protocols (Poloni et al 1997). While purity of selected CD34+ cells is in the range of 70% to 90%, the yield of the separation process is much lower. Efficiency of recovery protocols largely varies, yielding between 20% and 70% (Servida et al 1996; Almichi et al 1997; Mobest et al 1999; Querol et al 2000; Polouckova et al 2001, Flores-Guzman et al 2002). As such, enrichment of the cell population to near homogeneity is associated with loss of about half of the target cells. Thus, it would be advantageous to have an alternative to methods for expansion using selected populations of hematopoietic stem cells.

The mononuclear cell fraction is a highly heterogeneous cell population found within the marrow, cord blood and peripheral blood, including, among others, all the CD34+ cells. However, attempts to expand hematopoietic stem cells using mononuclear cells as the source cell populations described in the literature reported that the harvested cells were mostly early committed progenitors, rather than hematopoietic stem cells and that growth conditions were based on use of a growth medium supplemented with a cocktail of cytokines (Koller et al 1993; Sandstrom et al 1995; Shpall et al, Biol of Blood and Marrow Transpl 2002; 8:368-76; McNiece et al 2004; Mao et al 2005), or growth on supportive two dimensional mesenchymal cell cultures (McNiece et al Cytotherapy 2004 6:311-317).

There is thus a widely recognized need for, and it would be highly advantageous to have, a method and apparatus for ex-vivo expansion and/or maintenance of transplantable hematopoietic stem cells from mononuclear cells, devoid of the above limitations, with superior results as is compared to the teachings of the prior art.

### SUMMARY OF THE INVENTION

While reducing the present invention to practice, methods of ex-vivo expansion of hematopoietic stem cells using a bioreactor or flow system seeded with mesenchymal cells were developed. Mononuclear cells cultivated with mesenchymal cells from a mesenchymal cell containing tissue on three dimensional porous carriers, to provide expanded hematopoietic stem cells for transplantation with high engraftment potential. While reducing to practice, the present invention shows that spatial cultures of mesenchymal cells can support significant expansion of hematopoietic stem cells without need for hematopoietic stem cells subpopulation preselection, and that the absolute expansion magnitude is greater when unselected mononuclear cells rather than CD34+ selected cells are used for expansion. The present invention combines three dimensional scaffold methodology with flow-through and co-culture techniques and allows for the cultivation of primary mesenchymal cells on porous carriers to a high density closely mimicking the natural marrow environment. The present invention is capable of expanding both mesenchymal cells and hematopoietic stem cells to a large extent in an environment devoid of supplemented chemokines, cytokines and growth factors.

Thus, according to one aspect of the present invention there is provided a method of expanding and/or maintaining undifferentiated hematopoietic stem cells, the method comprising the steps of (a) obtaining unselected mononuclear cells; and (b) seeding the unselected mononuclear cells into a stationary phase plug-flow bioreactor in which a three dimensional stromal cell culture has been pre-established on a substrate in the form of a sheet, the substrate including a non-woven fibrous matrix forming a physiologically acceptable three-dimensional network of fibers, thereby expanding undifferentiated hematopoietic stem cells.

According to still further features in the described preferred embodiments the method further comprising the step of isolating the mononuclear cells.

According to still another aspect of the present invention there is provided a method of transplanting undifferentiated hematopoietic stem cells into a recipient, the method comprising the steps of (a) expanding the undifferentiated hematopoietic stem cells by (i) obtaining unselected mononuclear cells; and (ii) seeding the mononuclear cells into a stationary phase plug-flow bioreactor in which a three dimensional stromal cell culture has been pre-established on a substrate in the form of a sheet, the substrate including a non-woven fibrous matrix forming a physiologically acceptable three-dimensional network of fibers, thereby expanding undifferentiated hematopoietic stem cells; and (b) transplanting the undifferentiated hematopoietic stem cells resulting from step (a) in the recipient.

According to still further features in the described preferred embodiments the method further comprising the step of isolating the mononuclear cells prior to step (b).

According to further features in preferred embodiments of the invention described below, the mononuclear cells are isolated from a tissue selected from the group consisting of cord blood, peripheral blood, mobilized peripheral blood and bone-marrow.

According to further features in preferred embodiments of the invention described below, the mesenchymal cells are isolated from a source selected from the group consisting of umbilical cord cells, bone cells, placental cells, bone marrow cells and adipose tissue cells.

According to yet further features in the described preferred embodiments the mesenchymal cells are adherent cells of a mesenchymal tissue.

According to yet further features in the described preferred embodiments the mesenchymal cells are mesenchymal stem cells.

According to still further features in the described preferred embodiments the mononuclear stem cells and stromal cells of the stromal cell culture share common HLA antigens.

According to still further features in the described preferred embodiments the mononuclear cells and stromal cells of the stromal cell culture are from a single individual.

According to still further features in the described preferred embodiments the mononuclear cells and stromal cells of the stromal cell culture are from different individuals.

According to still further features in the described preferred embodiments the mononuclear cells and stromal cells of the stromal cell culture are from the same species.

According to still further features in the described preferred embodiments the mononuclear cells and stromal cells of the stromal cell culture are from different species.

According to still further features in the described preferred embodiments stromal cells of the stromal cell culture are grown to a density of at least 1×10⁶ cells per a cubic centimeter of the substrate.

According to still further features in the described preferred embodiments stromal cells of the stromal cell culture are grown to a density of at least 5×10⁶ cells per a cubic centimeter of the substrate.

According to still further features in the described preferred embodiments stromal cells of the stromal cell culture are grown to a density of at least 10⁷ cells per a cubic centimeter of the substrate.

According to still further features in the described preferred embodiments the step of seeding the mononuclear cells into the stationary phase plug-flow bioreactor is effected while flow in the bioreactor is shut off for at least 10 hours following the seeding.

According to still further features in the described preferred embodiments the fibers form a pore volume as a percentage of total volume of from 40% to 95% and a pore size of from 10 microns to 100 microns.

According to still further features in the described preferred embodiments the matrix is made of fiber selected from the group consisting of flat, non-round, and hollow fibers and mixtures thereof, the fibers being of from 0.5 microns to 50 microns in diameter or width.

According to still further features in the described preferred embodiments the matrix is composed of ribbon formed fibers having a width of from 2 microns.

According to still further features in the described preferred embodiments the matrix having a pore volume as a percentage of total volume of from 60% to 95%.

According to still further features in the described preferred embodiments the matrix has a height of 50-1000 μm.

According to still further features in the described preferred embodiments the material of the matrix is selected from the group consisting of polyesters, polyalkylenes, polyfluorochloroethylenes, polyvinyl chloride, polystyrene, polysulfones, cellulose acetate, glass fibers, and inert metal fibers.

According to still further features in the described preferred embodiments the matrix is in a shape selected from the group consisting of squares, rings, discs, and cruciforms.

According to still further features in the described preferred embodiments the matrix is coated with poly-D-lysine.

According to still further features in the described preferred embodiments the stromal cells comprise stromal cells of primary culture.

According to still further features in the described preferred embodiments the stromal cells comprise stromal cells of a cell line.

According to still further features in the described preferred embodiments a rate of the continuous flow is in a range of 0.1 to 25 ml/minute.

According to still further features in the described preferred embodiments a rate of the continuous flow is in a range of 1 to 10 ml/minute.

The present invention successfully addresses the shortcomings of the presently known configurations by providing more effective means for expanding/maintaining undifferentiated hematopoietic stem cells.

Implementation of the method and bioreactor of the present invention may involve performing or completing selected tasks or steps manually, automatically, or a combination thereof.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIG. 1 is a graphic representation of the growth of mesenchymal cells seeded onto porous polyester carriers in a flow system bioreactor. Cells from collagenase-treated adipose tissue (PLA) were isolated and seeded onto porous polyester carriers at load of 30,000 cells per carrier. At the indicated time points (closed squares), up to 45 days, carriers were removed from the bioreactor and counted as detailed hereinbelow;

FIG. 2 is a graphic representation of the growth of placenta-derived mesenchymal cells (PLC) on polystyrene carriers in a plug-flow bioreactor system. Mesenchymal stem cells derived from placenta were seeded onto the three dimensional carriers at a load of 10-15,000 cells per carrier. Each curve represents a separate experiment. Carriers were removed from the bioreactor after 6 and 12 days and the cells were counted as detailed hereinbelow. At 12 days of culture the cell density in the cultures was 150-250,000 cells per carrier;

FIG. 3 is a graphic representation of the growth of bone marrow-derived stroma cells grown onto porous polyester carriers in a flow bioreactor. Mesenchymal stem cells derived from bone marrow were seeded onto the three dimensional carriers at a load of 75,000 cells per carrier. Carriers were sampled from the bioreactor weekly for up to 50 days (closed squares). The cells were counted as detailed hereinbelow. After 50 days the cells reach a density of nearly 1,400,000 cells/carrier;

FIGS. 4a-4h are photomicrographs demonstrating the propagation to high densities of spatial cultures of mesenchymal cells in a flow bioreactor. FIGS. 4a-4c are photos of a Geimsa stain of the cells growth on the porous carrier, taken at 7 (4a), 14 (4b) and 21 (4c) days in culture. FIGS. 4d and 4e are histological preparations of cells grown on the porous carriers taken at 7 (4d) and 40 (4e) days in culture. FIGS. 4f-4h are SEM images of mesenchymal cells grown on the porous carriers, taken at 0 (4f), 20 (4g) and 40 (4h) days in culture. Note that the cell growth is not restricted to the scaffold surface but fills all the inner volume;

FIGS. 5a-5c are immunohistological sections showing the expansion of early hematopoietic stem cells on mesenchymal cells grown on 3-D carriers in a flow system. Hematopoietic stem cells were plated onto high-density stroma cell cultures grown on 3-D carriers in a flow system. Carriers were harvested after 7 days, fixed and sectioned, immuno-stained with CD34 monoclonal Ab and visualized using peroxidase-conjugated second antibody. FIGS. 5a-5c are representative sections illustrating the interaction between the early hematopoietic cells (CD34⁺, arrows) and the 3-D mesenchymal cells culture microenvironment;

FIGS. 6a-6d are a graphic representation illustrating superior expansion of hematopoietic stem cells grown from unselected mononuclear cells as compared to a purified CD34+ cell fraction. Human primary bone marrow-derived stroma cells were seeded and grown on 3-D carriers in the flow system to high density, seeded with either unselected mononuclear cells or the CD34+ fraction, and grown for 21 days. CD34+(FIG. 6a) and CD34+CD38− (FIG. 6b) cells were sampled and analyzed by flow cytometry every 7 days. Note the consistently superior fold expansion of CD34+(FIG. 6c) and CD34+CD38− (FIG. 6d) from the unselected mononuclear cell fraction (red lines and columns). Results represent the Mean+SD of 3-6 representative carriers;

FIGS. 7a-7b are a graphic representation of FACS analysis illustrating superior expansion of hematopoietic stem cells grown from unselected mononuclear cells as compared to a purified CD34+ cell fraction. Spatial cultures of bone-marrow derived mesenchymal cells, grown as in FIGS. 6a-6d, were seeded with either unselected mononuclear cells (MNC) (FIG. 7a) or a hematopoietic stem cell (CD34+) fraction (FIG. 7b). FACS analysis at 14 days culture of CD34+(quadrants A2+A4) and CD34+/CD38− (quadrants A4) cells indicates superior growth and expansion with the mononuclear cells (FIG. 7a);

FIG. 8 is a graphic representation of superior expansion and growth of hematopoietic stem cells grown from unselected mononuclear cells in a flow system. Human bone marrow-derived stroma cells were seeded and grown to high density on 3-D carriers in the flow system. The carriers were then seeded with either CD34+ selected hematopoietic stem cells or unselected mononuclear cells. Cultivation was allowed to proceed for an additional period of 7-21 days, and CD34+(selected hematopoietic stem cells=solid squares, unselected mononuclear cells=solid diamonds) and CD34+38− (selected hematopoietic stem cells=solid triangles; unselected mononuclear cells=Xs) markers were analyzed weekly by flow cytometer;

FIGS. 9a-9b are histograms illustrating the superior expansion of unselected mononuclear cells in a bone-marrow derived flow system, as in FIG. 8. Human bone marrow-derived stroma cell were seeded and grown to high density on 3-D carriers in the flow system. The carriers were then seeded with either unselected mononuclear cells (A-MNC) or CD34+ selected hematopoietic stem cells (B-HSC). Cultivation was allowed to proceed for an additional period of 7-21 days (yellow=7 days, blue=14 days, red=21 days). CD34+, CD45+ and CD34+38− cells were analyzed every 7 days by flow cytometer (FIG. 9a=CD45+CD34+; FIG. 9b=CD45+CD34+CD38−);

FIGS. 10a and 10b are a graphic representation of superior growth and expansion of CD34+ and CD43+CD38− from unselected mononuclear cells grown in co-culture with umbilical cord vein-derived mesenchymal cells in a flow system. Human cord vein-derived stroma cells were seeded and grown to high density on 3-D carriers in the flow system. The carriers were then seeded with either unselected mononuclear cells (MNC) or CD34+ selected hematopoietic stem cells (34+). Cultivation was allowed to proceed for an additional period of 7-21 days (blue triangles=mononuclear cells, red triangles=selected hematopoietic stem cells (CD34+) cells). CD34+, CD45+ and CD34+38− cells were analyzed every 7 days by flow cytometry (FIG. 10a=CD45+CD34+; FIG. 10b=CD45+CD34+CD38−);

FIG. 11 is a schematic depiction of an exemplary plug-flow bioreactor 20 which served while reducing the present invention to practice; 1—medium reservoir; 2—gas mixture container; 3—gas filters; 4—injection points; 5—plug or container of plug flow bioreactor 20; 6—flow monitors; 6a—flow valves; 7—conditioned medium collecting/separating container; 8—container for medium exchange; 9—peristaltic pump; 10—sampling point; 11—container for medium exchange; 12—O₂ monitor; 14—steering device; PH—pH probe.

### DESCRIPTION OF THE PREFERRED EMBODIMENTS

The present invention is of methods and bioreactor for hematopoietic stem cell expansion/maintenance which can be used for transplantation in a recipient or for other purposes as if further detailed hereinunder. Specifically, the present invention is of a three dimensional stromal cell plug flow bioreactor for the maintenance and/or expansion of hematopoietic stem cells from mononuclear cell cultures, which can be used in a variety of applications.

The principles and operation of the present invention may be better understood with reference to the drawings and accompanying descriptions.

Before explaining at least one embodiment of the invention in detail, it is to be understood that the invention is not limited in its application to the details of construction and the arrangement of the components set forth in the following description or illustrated in the drawings. The invention is capable of other embodiments or of being practiced or carried out in various ways. Also, it is to be understood that the phraseology and terminology employed herein is for the purpose of description and should not be regarded as limiting.

In the developing medical world, there is a growing need for stem cells, and more specifically for hematopoietic stem cells and for stromal stem cells (also termed “mesenchymal stem cells”), for clinical and research purposes. Mesenchymal stem cells are used for support of hematopoietic stem cell transplantation and engraftment and also for curing a growing number of conditions e.g., heart diseases, bone marrow deficiencies, neuronal related diseases, and conditions which require organ or tissue transplantation.

U.S. Pat. No. 6,911,201 to Merchav et al discloses a method of growing and expanding undifferentiated transplantable hematopoietic cells by culturing the selected populations of hematopoietic cells (CD34+ cells) on spatially organized carriers mimicking the bone marrow microstructure have been utilized. These carriers are capable of supporting the growth and prolonged maintenance of stromal cells in a plurality of selected bioreactor systems under culture conditions devoid of supplemented cytokines. The latter systems include, but are not limited to plug-flow and roller bottle bioreactors. Practically, stroma cells are cultivated onto spatial, porous biodegradable or non-biodegradable carriers made of non-woven fabric matrix, enabling the propagation of large cell numbers in a relatively small volume. The stroma cells cultured in these systems retain the capacity of to promote maintenance and expansion of transplantable human hematopoietic stem cells.

U.S. patent application Ser. Nos. 11/102,625, 11/102,654, 11/102,623 and 11/102,625, to Merchav et al, further disclose that conditioned medium from the three dimensional stromal culture as taught in U.S. Pat. No. 6,911,201 can effectively support expansion and growth of hematopoietic stem cells in an undifferentiated state.

However, hematopoietic stem cell expansion in U.S. Pat. No. 6,911,201 and U.S. patent application Ser. Nos. 11/102,625, 11/102,654, 11/102,623 and 11/102,625 was initiated from a cell population enriched for hematopoietic stem cells (CD34+), and not from total, unselected-mononuclear cells.

While reducing the present invention to practice, it was surprisingly uncovered that non-selected mononuclear cells from various hematopoietic sources (cord blood, bone marrow and peripheral blood) could serve as the founding cell pool of expandable hematopoietic stem cells. Using these cells as the root population for hematopoietic stem cell proliferation overcomes the present need for costly and complex purification of the founding cell population, and the significant loss of target cell population associated with the stem cell selection process.

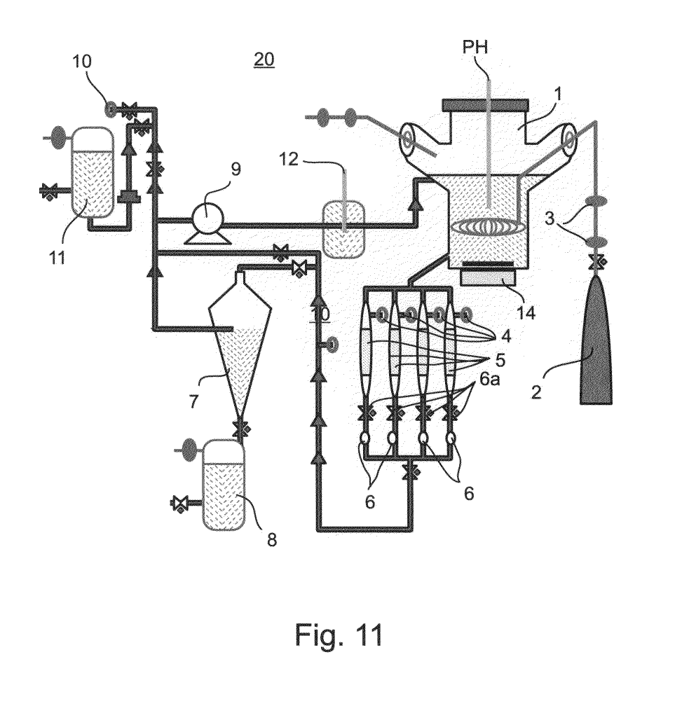

Thus, according to one aspect of the present invention there is provided a method of expanding/maintaining undifferentiated mononuclear-derived hematopoietic stem cells. The method according to this aspect of the present invention is effected by seeding unselected mononuclear cells into a stationary phase plug-flow bioreactor, an example of which is depicted in FIG. 11 along with reference numerals, in which a three dimensional stromal cell culture (e.g., stromal cell line or primary stromal cell culture), has been pre-established on a substrate in the form of a sheet, the substrate including a non-woven fibrous matrix forming a physiologically acceptable three-dimensional network of fibers, thereby, as is further described above and exemplified in the Examples section that follows, expanding/maintaining undifferentiated hematopoietic stem cells.

As used herein in the specification and in the claims section that follows, the phrase “undifferentiated hematopoietic stem cells” refers to uncommitted hematopoietic cells.

Undifferentiated hematopoietic stem cells and early committed cells are CD34+ cells. Thus, the phrase “obtaining undifferentiated hematopoietic stem cells” and its equivalent phrase “undifferentiated hematopoietic stem cells are obtained” both refer to the obtainment of either isolated undifferentiated hematopoietic stem cells, or a population of CD34+ cells which contain undifferentiated hematopoietic stem cells.

As used herein in the specification and in the claims section that follows, the terms “expanding” and “expansion” refer to substantially differentiation-less cell growth, i.e., increase of a cell population without differentiation accompanying such increase.

As used herein in the specification and in the claims section that follows, the terms “maintaining” and “maintenance” refer to substantially differentiation-less cell renewal, i.e., substantially stationary cell population without differentiation accompanying such stationarity.

As used herein the term “differentiation” refers to an irreversible transition from relatively generalized to specialized kinds during development. Cell differentiation of various cell lineages is a well documented process and requires no further description herein.

As used herein, the term “mononuclear cells”, “unselected mononuclear cells” or “unselected mononuclear cells population” is defined as a population or sample of mononuclear cells including the entire complement of white blood cells present in a blood sample, comprising a majority fraction of the cells having committed hematopoietic precursor cells, and an uncommitted minority fraction having pluripotent hematopoietic cells, which population has not undergone selection for hematopoietic stem cells. Preferably, the mononuclear cells comprise a population of cells in which the uncommitted minority fraction having pluripotent hematopoietic cells is 0.01% to 1%, more preferably 1% than 2%, even more preferably 2% to 5%, yet more preferably 5% to 10%, yet more preferably 10% to 30%, and more preferably 30% to 49% of the total mononuclear cells. Suitable unselected mononuclear cells can be from any source relatively rich in hematopoietic cells, such as cord blood, peripheral blood, placenta, bone marrow, etc. Methods for identification and isolation of mononuclear cells are well known in the art, such as density centrifugation.

The mononuclear fraction of blood, commonly isolated from the “buffy coat” of density gradient-separated whole blood, normally contains very few hematopoietic stem cells. In a healthy human being, the mononuclear comprise a mixture of hematopoietic lineages committed and differentiated cells (typically over 99% of the mononuclear cells are lineages committed cells) including, for example: Lineage committed progenitor cells CD34⁺CD33⁺ (myeloid committed cells), CD34⁺CD3⁺ (lymphoid committed cells) CD34⁺CD41⁺ (megakaryocytic committed cells) and differentiated cells—CD34⁻CD33⁺ (myeloids, such as granulocytes and monocytes), CD34⁻CD3⁺, CD34⁻CD19⁺ (T and B cells, respectively), CD34⁻CD41⁺ (megakaryocytes), and hematopoietic stem and early progenitor cells such as CD34⁺CD38⁻ (typically less than 1%).

As used herein, the phrase “hematopoietic committed cells” refers to differentiated hematopoietic cells that are committed to a certain hematopoietic cell lineage and hence can develop under physiological conditions substantially only to this specific hematopoietic lineage.

As used herein, the phrase “mensenchymal cell” is interchangeable with the phrase “stromal cell” or “mesenchymal stromal cell” and refers to a cell or cells derived from the mesodermal layer, e.g., mesenchymal stem. Mesenchymal cells originate from the mesodermal layer of embryonic cells during development, and are present in every organ including subcutaneous tissue, lungs, liver, and mesenchymal tissue such as bone, cartilage, fat, tendon, skeletal muscle and the stroma of bone marrow. Mesodermal cells can also be characterized, and isolated, by a number of prospective markers: presently, the presence of CD 73 and/or CD105 and/or CD166 and/or CD29 and/or CD90 and/or CD44, CD49b, SH(1), SH(2), SH(3), or SH(4) surface antigens, the absence of CD34, CD14, CD45, and HLA class 1, as well as superior adherence to plastic and multipotent differentiation potential, help to identify cells of mesenchymal lineage from various tissue sources (see Horowitz, Cytotherapy 2000, 2:387-88, and Lee et al, BBRC 2004; 320:273-78, and US Patent Application Nos. 20020058289 and 20040058397 to Thomas, et al).

According to a preferred embodiment, the mesenchymal cells are adherent cells obtained from a source selected from umbilical cord cells, placental cells, adipose tissue cells, bone cells and bone marrow cells. Methods of mesenchymal cell culture are well known in the art of cell culturing (see, for example, Friedenstein, et al, Exp Hematol 1976 4, 267-74; Dexter et al. J Cell Physiol 1977, 91:335-44; and Greenberger, Nature 1978 275, 752-4).

As used herein the term “ex-vivo” or “in vitro” refers to cells removed from a living organism and maintained or propagated outside the organism (e.g., in a test tube).

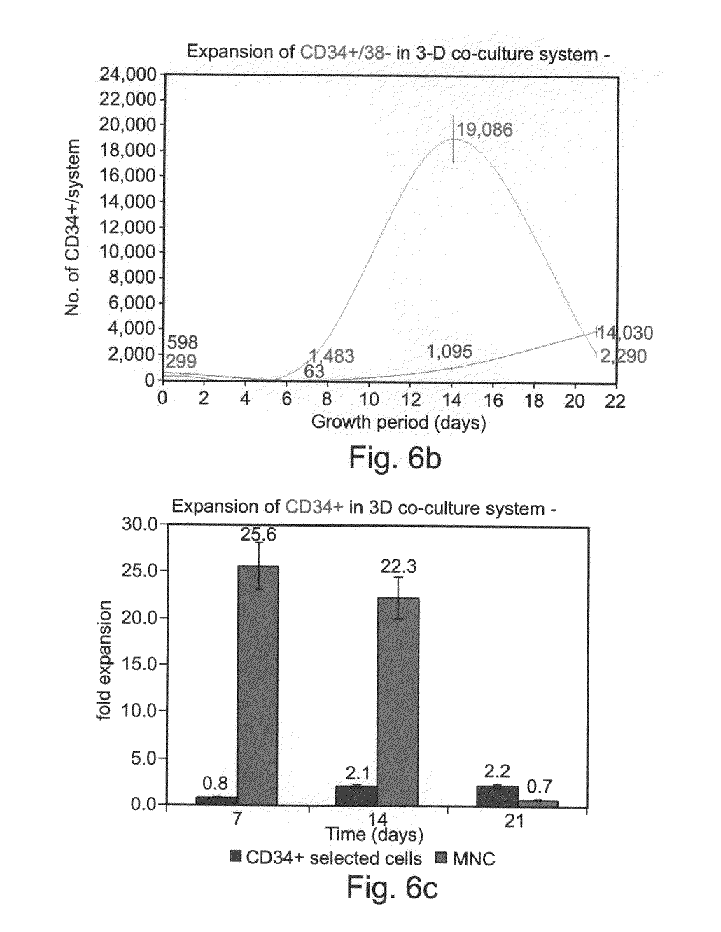

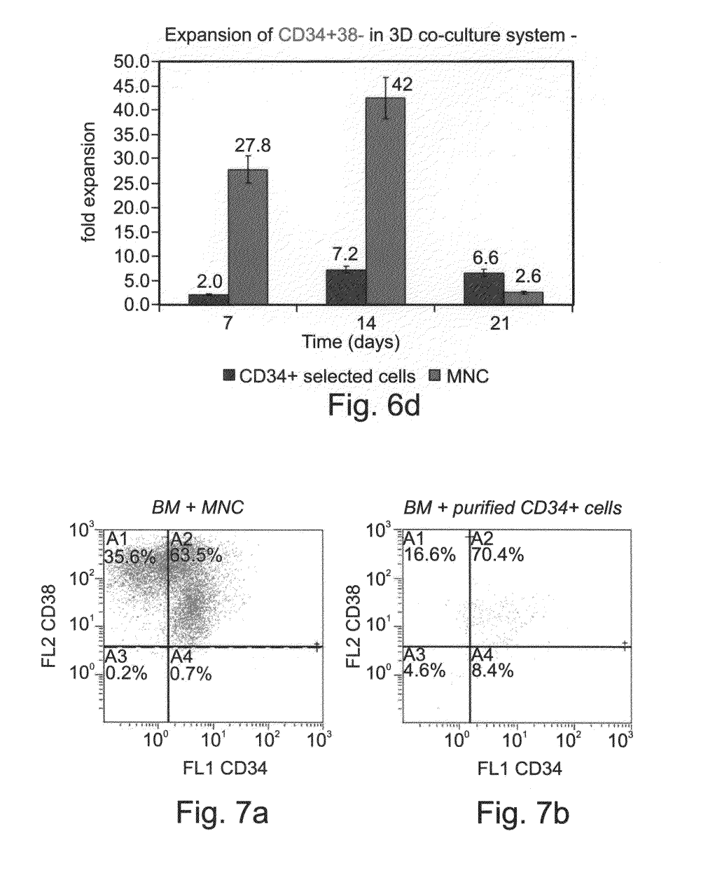

Expansion of hematopoietic stem cells from unselected mononuclear cells using a three dimensional plug flow bioreactor which closely mimics the bone marrow microenvironment and which is capable of supporting the growth and prolonged maintenance of marrow stromal cells is described herein. In the examples provided in the Example section that follows, the bioreactor was seeded with the adipose-, placenta-, cord blood- or bone marrow-derived mesenchymal cells, grown to high cell density, and then seeded with unselected human mononuclear cells (see FIGS. 6-10). In every case, expansion of the hematopoietic fraction (CD34+ or CD34+CD38−) in the mononuclear cells cultured on the mesenchymal cells was superior to that of the hematopoietic fraction of selected CD34+ cells grown in the same way, in short and long term cultures.

According to a preferred embodiment of the present invention the unselected mononuclear cells and stromal cells of the stromal cell culture share common HLA antigens. According to another preferred embodiment of the present invention the unselected mononuclear cells and the stromal cells of the stromal cell culture are from a single individual. Thus, separation of cells is not required in case of transplantation thereof to a recipient.

According to still another preferred embodiment of the present invention the unselected mononuclear cells and stromal cells of the stromal cell culture are from different individuals. For example, a future recipient of the undifferentiated hematopoietic stem cells and stromal cells can be used to provide the stromal cells, whereas the unselected mononuclear cells and stromal cells are from a donor selected according to HLA compatibility to donate such cells to the recipient. Thus, again, separation of cells is not required prior to transplantation.

According to another embodiment of the present invention the unselected mononuclear cells and stromal cells of the stromal cell culture are from the same species. However, according to still another preferred embodiment of the present invention the unselected mononuclear cells and stromal cells of the stromal cell culture are from different species.

The bioreactor described herein is unique in that it combines both three dimensional stromal cell cultures with a continuous flow system. Three dimensional mixed cell systems such as the system described in U.S. Pat. No. 6,911,201 clearly demonstrate the superior efficiency of growth of hematopoietic cells on three dimensional stromal cell cultures relative to monolayers, in the absence of continuous flow.

The three-dimensional plug-flow bioreactor described herein is capable of supporting the long-term growth of stromal cell lines, as well as primary marrow stromal cells from different sources. The use of stromal cells in the bioreactor is not only essential for the establishment of superior stromal-stem cell contact (via unique “niches” and cell-cell, cell-ECM interactions), but also for stromal cell production of known and novel soluble and membrane-bound cytokines. Stromal cells can facilitate the supplementation of such bioreactors with appropriate cytokines, by using genetically engineered cytokine-producing variant cells. For example, in such a manner, cytokine combinations specifically suited for growth and expansion of mononuclear cell cultures can be identified and provided. Bioreactor stromal cells can also be engineered to serve as retroviral packaging cell lines, enabling the efficient transduction of genetic material into stem cells, within the bioreactor itself. The use of various stromal cells in the bioreactor can also allow the selection of the most suitable substrate for purging of Ph-positive stem cells, the latter known for their lesser capacity for stromal cell adherence. Primary stromal cells have the advantage that they enable the establishment of “autologous” stromal-stem cell bioreactors, on which autologous or even cord blood stem cells can be expanded and which eliminate the need to remove stromal cells prior to transplantation.

Thus, expansion of the undifferentiated hematopoietic cells from unselected mononuclear cells is preferably performed in a three dimensional plug flow bioreactor.

Preferably, the bioreactor of the present invention employs a growth matrix that substantially increases the available attachment surface for the adherence of the stromal cells so as to mimic the mechanical infrastructure of bone marrow. When the matrix is used in sheet form, preferably non-woven fiber sheets, or sheets of open-pore foamed polymers, the preferred thickness of the sheet is about 50 to 1000 μM or more, there being provided adequate porosity for cell entrance, entrance of nutrients and for removal of waste products from the sheet. According to a preferred embodiment the pores having an effective diameter of 10 μm to 100 μm. Such sheets can be prepared from fibers of various thicknesses, the preferred fiber thickness or fiber diameter range being from about 0.5 μm to 20 μm, still more preferred fibers are in the range of 10 μm to 15 μm in diameter.

The structures of the invention may be supported by, or even better bonded to, a porous support sheet or screen providing for dimensional stability and physical strength.

Such matrix sheets may also be cut, punched, or shredded to provide particles with projected area of the order of about 0.2 mm² to about 10 mm², with the same order of thickness (about 50 to 1000 μm).

Further details relating to the fabrication, use and/or advantages of the growth matrix which was used to reduce the present invention to practice are described in U.S. Pat. Nos. 5,168,085, in particular, 5,266,476, and also 6,991,933, all of which are incorporated herein by reference.

As will readily be appreciated by the skilled artisan, the present invention provides expanded undifferentiated hematopoietic stem cell population which can be used in a variety of applications, such as, but not limited to: (i) expansion of human stem cells (of autologous or cord blood source) on recipient stroma, without the need for stromal-stem cell separation prior to transplantation; (ii) depletion of Ph+CML stem cells in an autologous setting via stromal-stem cell interactions; (iii) gene transfer into self-renewing stem cells within the bioreactor or following harvesting from the bioreactor.

As is shown in FIG. 11, according to yet an additional aspect of the present invention there is provided a bioreactor plug comprising a container 5, typically in the form of a column, having an outlet and an inlet and containing therein a substrate in the form of a sheet, the substrate including a non-woven fibrous matrix forming a physiologically acceptable three-dimensional network of fibers, the substrate supporting at least 1×10⁶ stromal cells/ml, preferably, at least 5×10⁶ cells/ml, most preferably at least 10⁷ cells/ml, of either stromal cell line or primary stromal cell culture, per cubic centimeter of the substrate.

It will be appreciated in this respect that the substrate may theoretically support up to 5×10⁷ cells per cubic centimeter thereof. Once sufficient cells have accumulated on the substrate, means such as irradiation can be employed to cease further cell growth, so as to control the exact number of cells supported by the substrate.

According to a presently preferred embodiment of the present invention the step of seeding the unselected mononuclear cells into the stationary phase plug-flow bioreactor is effected while flow in the bioreactor is shut off for at least 10 hours following such seeding, so as to enable the cells to anchor to the stromal cell covered matrix.

According to preferred embodiments of the present invention, culturing the stromal cells of the present invention is effected under continuous flow of the culture medium. Preferably the flow rate through the bioreactor is between 0.1 and 25 ml/minute, more preferably the flow rate is between 1-10 ml/minute.

The following descriptions provide insight with respect to preferred substrates which are used while implementing the present invention.

Thus, according to one embodiment the fibers of the substrate form a pore volume as a percentage of total volume of from 40 to 95% and a pore size of from 10 microns to 100 microns. According to another embodiment, the matrix making the substrate is made of fiber selected from the group consisting of flat, non-round, and hollow fibers and mixtures thereof, the fibers being of from 0.5 microns to 50 microns in diameter or width. According to still another embodiment, the matrix is composed of ribbon formed fibers having a width of from 2 microns. According to a further embodiment, the ratio of width to thickness of the fibers is at least 2:1. According to still a further embodiment, the matrix making the substrate having a pore volume as a percentage of total volume of from 60 to 95%. According to still another embodiment, the matrix has a height of 50-1000 μm, whereas stacks thereof are employed. According to yet another embodiment, the material of the matrix making the substrate is selected from the group consisting of polyesters, polyalkylenes, polyfluorochloroethylenes, polyvinyl chloride, polystyrene, polysulfones, cellulose acetate, glass fibers, and inert metal fibers. According to still another embodiment, the matrix is in a shape selected from the group consisting of squares, rings, discs, and cruciforms. According to still another embodiment, the matrix is coated with poly-D-lysine.

As demonstrated in the Examples section hereinbelow, significant expansion of hematopoietic stem cells from unselected mononuclear cell fraction was achieved in bioreactors, when co-cultured with mesenchymal and/or stromal cell cultures, without need for added cytokines or growth factors (see Example 2 hereinbelow, and FIGS. 6-10). Thus, according to the methods of the present invention, hematopoietic stem cell expansion from unselected mononuclear cells can be performed in a culture media without supplementation with exogenous cytokines and/or growth factors. Briefly, mononuclear cells isolated from Ficoll pellets of tissue samples (umbilical cord blood, bone marrow and peripheral blood) are suspended in artificial serum-free growth media, or media supplemented with 10% bovine serum, and seeded onto the pre-established stroma cells three dimensional cultures. Under these conditions, the seeded mononuclear cells can be expanded and provide superior hematopoietic stem cell expansion.

Following expansion, the now expanded undifferentiated hematopoietic stem cells can be isolated by a variety of affinity separation/labeling techniques, such as, but not limited to, fluorescence activated cell sorting and affinity separation via an affinity substrate. Affinity molecules which can be used to implement such isolation methods include anti-CD34 antibodies, for example, which bind CD34+ cells.

According to still another aspect of the present invention there is provided a method of transplanting expanded undifferentiated hematopoietic stem cells into a recipient. The method according to this aspect of the present invention is effected by implementing the following method steps. First, the undifferentiated hematopoietic stem cells are expanded from unselected mononuclear cells by any of the methods described above. Second, the undifferentiated hematopoietic stem cells resulting from the first step are transplanted in the recipient.

Transplantation is generally effected using methods well known in the art, and usually involves injecting or introducing the hematopoietic stem cells into the subject using clinical tools well known by those skilled in the art (U.S. Pat. Nos. 6,447,765, 6,383,481, 6,143,292, and 6,326,198).

For example, introduction of the expanded hematopoietic stem cells of the present invention can be effected locally or systematically via intravascular administration, including intravenous or intraarterial administration, intraperitoneal administration, and the like. Cells can be injected into a 50 mol Fenwall infusion bag using sterile syringes or other sterile transfer mechanisms. The cells can then be immediately infused via IV administration over a period of time, such as 15 minutes, into a free flow IV line into the patient. In some embodiments, additional reagents such as buffers or salts may be added as well. The composition for administration must be formulated, produced and stored according to standard methods complying with proper sterility and stability.

Stem cell dosages can be determined according to the prescribed use. In general, in the case of parenteral administration, it is customary to administer from about 0.01 to about 5 million cells per kilogram of recipient body weight. The number of cells used will depend on the weight and condition of the recipient, the number of or frequency of administrations, and other variables known to those of skill in the art. After administering the cells into the subject, the effect of the treatment may be evaluated, if desired, as known in the art. The treatment may be repeated as needed.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

### EXAMPLES

Reference is now made to the following examples, which together with the above descriptions, illustrate the invention in a non limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### Example 1

### Materials and Experimental Methods

Bioreactor: The bioreactor used in accordance with the teachings of the present invention was constructed in accordance with the design described in FIG. 11. The glassware was designed and manufactured by Pluristem, Inc. (Israel) and connected by silicone tubing (Degania, Israel). The carriers were rotated overnight in phosphate buffered saline (PBS; Beit Ha'Emek Industries, Israel) without Ca⁺² and Mg⁺², followed by removal of the PBS and released debris. Each column was loaded with 10-30 ml packed carrier. The bioreactor was filled with PBS-Ca—Mg, all outlets were sealed and the system was autoclaved (120° C., 30 minutes). The PBS was removed via container [8] and the bioreactor was circulated in a 37° C. incubator with 300 ml Dulbecco's high-glucose medium (DMEM; GIBCO BRL) containing 10% heat-inactivated fetal calf serum (FCS; Beit Ha'Emek Industries, Israel) and a Pen-Strep-Nystatin mixture (100 U/ml:100 μg/ml:1.25 μn/ml; Beit Ha'Emek), for a period of 48 hours. Circulating medium was replaced with fresh DMEM containing the above +2 mM L-glutamine (Beit Ha'Emek).

Stromal cells: Stromal cell lines were maintained at 37° C. in DMEM supplemented with 10% FCS, in a fully humidified incubator of 5% CO₂ in air. Cells were grown in tissue culture flasks (Corning) and were split by trypsinization upon reaching confluence.

Primary human marrow stromal cultures were established from aspirated sternal marrow of hematologically healthy donors. Briefly, marrow aspirates were diluted 3-fold in Hank's Balanced Salts Solution (HBSS; GIBCO BRL) and were subject to Ficoll-Hypaque (Robbins Scientific Corp. Sunnyvale, Calif.) density gradient centrifugation. Marrow mononuclear cells (<1.077 gm/cm³) were collected, washed 3 times in HBSS and resuspended in long-term culture (LTC) medium, consisting of DMEM supplemented with 12.5% FCS, 12.5% horse serum (Beit Ha'Emek, Israel), Cells were incubated in 25 ml tissue culture flasks (Corning) for 3 days at 37° C. (5% CO₂) and then at 33° C. (idem) with weekly culture refeeding. Stromal cells from individual donors were employed for each bioreactor.

Placenta derived stromal cells—Inner parts of a full-term delivery placenta (Bnei Zion medical center, Haifa, Israel) are cut under sterile conditions, washed 3 times with Hank's Buffer and incubated for 3 h at 37° C. with 0.1% Collagenase (1 mg collagenase/ml tissue). Using gentle pipeting, suspended cells are then washed with DMEM, seeded in 75 cm² flasks and incubated at 37° C. in a tissue culture incubator under humidified condition with 5% CO₂. After the purification process, cells are allowed to adhere to plastic surface for 72 hours after which the media is changed every 3 to 4 days. At 60-70% confluence (usually 10-12 days), the cells are detached from the growth flask using 0.25% trypsin-EDTA and seeded into new flasks.

Adipose derived stromal cells—cells were collected from adipose tissue using Collagenase and grown in DMEM supplemented with 10% FCS Streptomycin-Nystatin mixture and 0.1 mM of L-glutamin. At 40-60% confluence, the cells were detached with trypsin-EDTA and were then implanted (1000-10000 cells/cm2) and grown in a controlled tissue culture incubator under humidified conditions (5% CO₂; 37° C.), with routine examination for viability, shape, growth rate and sterility. Following 2-12 passages, when cells reached an adequate amount, cells were collected for analysis or for culturing in bioreactors.

For three dimensional and monolayer studies, primary stromal cell cultures were split by trypsinization (0.25% Trypsin and EDTA in Puck's Saline A; Beit Ha'Emek) every 10 days, to allow sufficient stromal cell expansion. For LTC-IC and CAFC (see below), stromal cells were irradiated (1500 cGy) using a ¹³⁷Cs source, cultures were maintained at 33° C. in LTC medium.

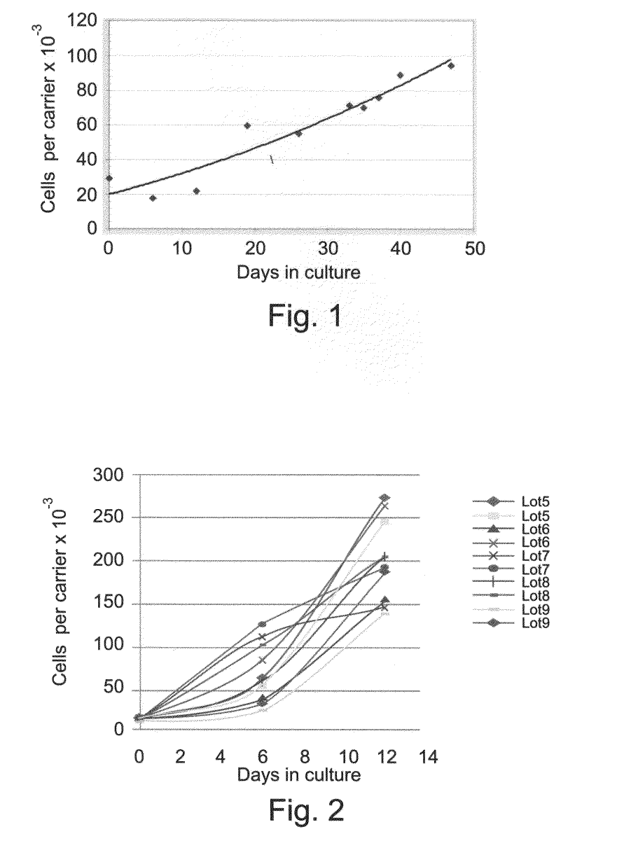

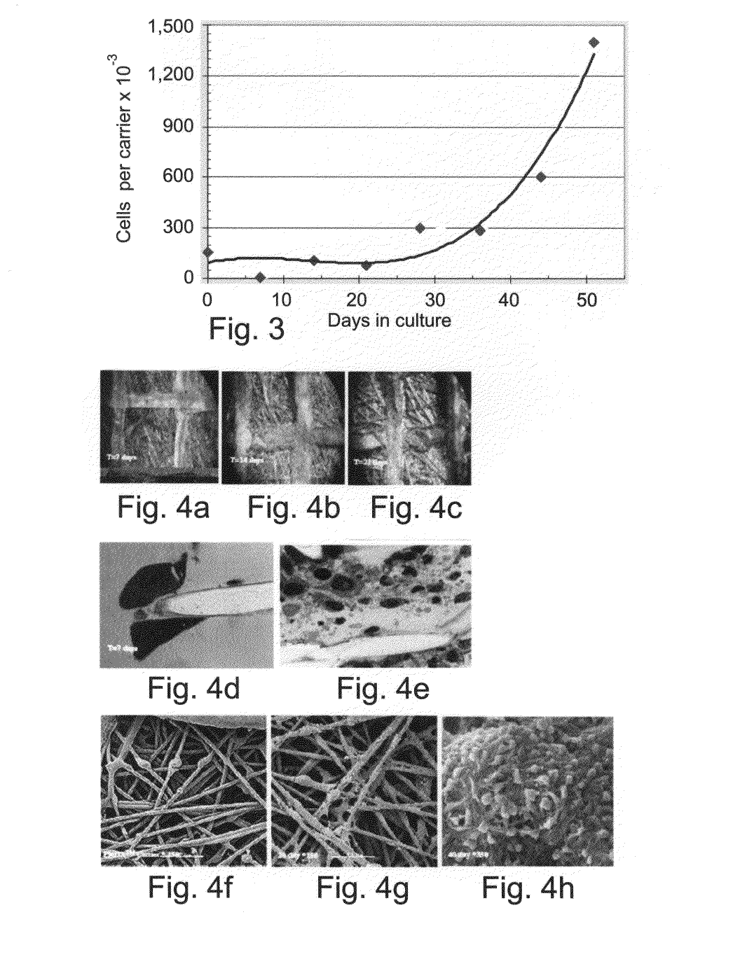

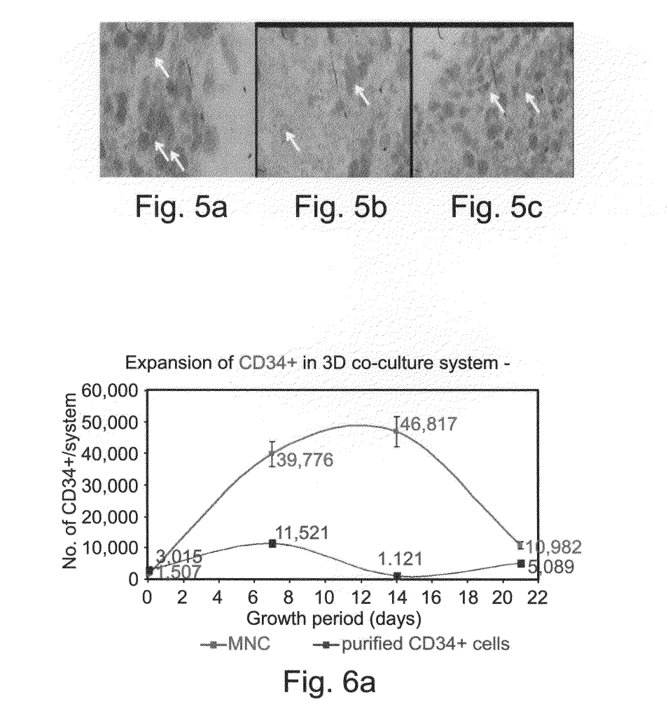

Seeding of stromal cells: Confluent cultures of stromal cell lines or 5-week primary marrow stromal cells were trypsinized and the cells washed 3 times in HBSS, resuspended in bioreactor medium (see above), counted and seeded at 10⁶ cells/ml in 10 ml volumes via an injection point ([4], FIG. 1) onto 10 ml carriers in the glass column of the bioreactor. Immediately following seeding, circulation was stopped for 5-16 hours to allow the cells to settle on the carriers. Stromal cell growth in the bioreactor was monitored by removal of carriers and cell enumeration by the MTT method (56). When stromal cells were confluent, medium was replaced with growth medium, for continued studies (preparation of SCM, stem cell seeding).

Hematopoietic/mesenchymal Cells—Bone marrow, placenta, cord umbilical vein, adipose tissue (from liposuction) and cord blood samples were obtained from Rambam Medical Center (Haifa, Israel), Laniado (Natania, Israel) and from Bnei-Zion Medical Center (Haifa, Israel) under local IRB approvals.

Mesenchymal cells—Mesenchymal cells from the adherent fraction of primary bone marrow cells, human bone marrow, fat samples or placenta were grown at 37° C. in basic DMEM medium, containing 10% heat-inactivated FCS, Penicillin-Streptomycin-Nystatin mixture and 0.1 mM of L-glutamin in a fully humidified incubator at 5% CO₂ in air. Cells are grown in tissue culture flasks and are dissociated by trypsinization upon reaching 60%-80% confluence. Under these conditions, the cells were able to proliferate for a period of more than one month. The mesenchymal cells were characterize by the presence of one or more of a panel of membrane markers like: CD29, CD44, CD73, CD90, CD105, CD166 and HLA class I, and the absence of expression of hematopoietic membrane markers like CD34, CD45 and CD14.

Mesenchymal cell three-dimensional cultures—Mesenchymal cells from individual donors were employed for each bioreactor. For three-dimensional and monolayer studies, primary mesenchymal cultures were dissociated by trypsinization. The mesenchymal cells were seeded onto porous carriers made of a non-woven fabric matrix of polyester, enabling the propagation of large cell numbers in a relatively small volume within the bioreactor system. The bioreactor is a continuous flow system in which the pH; dissolved oxygen; flow rate and temperature are controlled. Cultures were periodically sampled during a 50 days cultivation period.

Hematopoietic cells—Human hematopoietic stem cells, CD34+ cells and MNC samples were obtained from placental and umbilical cord in heparinized tubes. MNC samples were separated using Ficoll-Paque solution (density: 1.077 g/cm³). CD34+ cells were obtained from MNC fraction after immuno-magnetic separation using the CD34 midi-MACS selection kit (Miltenyi Biotec; Bergisch Gladbach, Germany). Hematopoietic stem cells were analyzed by the Beckman-Coulter FC-500 flow cytometer and characterize by the membrane markers CD34, CD38 and CXCR4.

Hematopoietic/mesenchymal cells three-dimensional co-cultures. In order to create hematopoietic/mesenchymal cell co-cultures, the hematopoietic cells (selected, CD34+ or unselected mononuclear cells) were seeded onto carriers pre-established with stroma cell/mesenchymal cells as described herein. Upon addition to the bioreactor, medium supply was suspended to enable contact with the mesenchymal cells. Co-culture were further cultivated in the flow bioreactor systems. In the examples provided in the Example section that follows, bioreactors containing three-dimensional cultures of mesenchymal cells from bone marrow, placenta or cord vein blood were seeded with cord blood-derived mononuclear and CD34+ cell. Cultures were allowed to grow in co-cultures conditions for additional period of up to 21 days. Mononuclear or CD34+ cell seeded-stromal cell carriers were removed for control studies in the absence of medium exchange. Co-cultures were maintained in growth media base on basic DMEM medium containing 10% heat-inactivated FCS, Penicillin-Streptomycin-Nystatin mixture and 0.1 mM of L-glutamin medium, without cytokines addition. At various times (up to 21 days), nonadherent cells were collected from circulating culture medium via a container. Adherent cells were collected via sequential trypsinization and exposure to EDTA-based dissociation buffer (GIBCO BRL), followed by gentle pipetting of the cells. Circulating and carrier-isolated hematopoietic cells were washed, counted and assayed separately for CD34, CD38 and CXCR4 by flow cytometry. Output assays can also include SRC, CAFC and LTC-IC.

Isolation of mononuclear cells and CD34+ cells: Umbilical cord blood samples taken under sterile conditions during delivery were fractionated on Ficoll-Hypaque and buoyant (<1.077 gr/cm³) mononuclear cells collected. Cells from individual CB samples were pooled, incubated with anti-CD34 antibodies and isolated by midi MACS (Miltenyi Biotech).

Stromal-hematopoietic stem cell cocultures: Isolated, pooled CB CD34+ cells were seeded at equivalent numbers (about 5×10⁵) onto monolayer or bioreactor containing equivalent densities of confluent stromal cells. Upon addition to the bioreactor, medium flow was stopped for 16 hours to enable contact with stromal cells and was re-initiated at a rate of 0.1-1.0 ml per minute. CD34+ cell seeded-stromal cell carriers were removed for control studies in the absence of medium exchange. Co cultures were maintained in growth medium, with or without cytokines. At various times (up to 4 weeks), nonadherent cells were collected from monolayer supernatants or from circulating culture medium via a container ([8], FIG. 1). Adherent cells were collected via sequential trypsinization and exposure to EDTA-based dissociation buffer (GIBCO BRL), followed by gentle pipetting of the cells. To avoid the presence of stromal cells in the resulting suspension, the cells were resuspended in HBSS+10% FCS and were subjected to a 60 minutes adhesion procedure in plastic tissue culture dishes (Corning), at 37° C. Circulating and carrier-isolated hematopoietic cells were washed, counted and assayed separately for CD34+/38−/CXCR4+ by flow cytometry. Output assays can also include SRC, CAFC and LTC-IC.

Flow Cytometry: Cells were incubated at 4° C. for 30 minutes with saturating concentrations of monoclonal anti-CD34+PerCP (Beckton-Dickinson), anti-CXCR4-fluorescein isothiocyanate (FITC, R&D systems) and -phycoerythrin (PE, Beckton-Dickinson) antibodies. The cells were washed twice in ice-cold PBS containing 5% heat-inactivated FCS and resuspended for three-color flow cytometry on a FC 500 (Beckman Coulter).

### Experimental Results

### Example 1

### Bioreactor System

The bioreactor system employed while reducing the present invention to practice is depicted in FIG. 11. It contained four parallel plug flow bioreactor units [5]. Each bioreactor unit contained 1 gram of porous carriers (4 mm in diameter) made of a non woven fabric matrix of polyester (58). These carriers enable the propagation of large cell numbers in a relatively small volume. The structure and packing of the carrier have a major impact on oxygen and nutrient transfer, as well as on local concentrations and released stromal cell products (e.g., ECM proteins, cytokines, 59). The bioreactor was maintained in an incubator of 37° C.

The flow in each bioreactor was monitored [6] and regulated by a valve [6a]. Each bioreactor contains a sampling and injection point [4], allowing the sequential seeding of stromal and mononuclear or hematopoietic cells. Culture medium was supplied at pH 7.0 [13] from a reservoir [1]. The reservoir was supplied by a filtered [3] gas mixture containing air/CO₂/O₂ [2] at differing proportions in order to maintain 5%-40% dissolved oxygen at exit from the column, depending on cell density in the bioreactor. The O₂ proportion was suited to the level of dissolved O₂ at the bioreactor exit, as was determined by a monitor [12]. The gas mixture was supplied to the reservoir via silicone tubes. The culture medium was passed through a separating container [7] which enabled collection of circulating, nonadherent cells. Circulation of the medium was obtained by means of a peristaltic pump [9] operating at a rate of 0.1-3 ml/minute. The bioreactor units were equipped with an additional sampling point [10] and two containers [8, 11] for continuous medium exchange at a rate of 10-100 ml/day. The use of few parallel bioreactor units enables periodic dismantling for purposes such as cell removal, scanning electron microscopy, histology, immunohistochemistry, RNA extraction, etc.

### Example 2

### Establishment of Three-Dimensional Mesenchymal/Stromal Cell Cultures in the Bioreactor

Cells of divergent origins were used for establishing the mesenchymal/stromal cell culture. Adipose cells, placental derived cells and bone marrow derived cells were seeded onto the polyester carriers as described hereinabove. Adipose tissue, seeded at a load of 30,000 cells per carrier, populated the carriers and proliferated to 100,000 cells per carrier at 45 days (FIG. 1). Placenta derived cells, prepared as described hereinabove, grew from less than 25,000 cells per carrier at seeding in the plug flow bioreactor, to 150-250,000 cells per carrier at 14 days in culture (FIG. 2). Bone marrow derived cells, loaded on the carriers at less than 75,000 cells per carrier, grew to a density of 1,500,000 cells per carrier after 50 days culturing as described hereinabove.

FIGS. 4a-4h demonstrate the propagation to high densities of the three-dimensional cultures of mesenchymal cells in a flow bioreactor. Photos taken at 7 (FIG. 4a), 14 (FIG. 4b), 21 (FIG. 4c) days in culture, histological preparations of cells grown on the porous carriers at 7 (FIG. 4d) and 40 (FIG. 4e) days in culture, and SEM images of mesenchymal cells grown on the porous carriers, taken at 0 (FIG. 4f), 20 (FIG. 4g) and 40 (FIG. 4h) days in culture all show that the cell growth on the carriers is not restricted to the scaffold surface but fills all the inner volume.

Thus, stromal cells of diverse origin can efficiently establish a high density, three-dimensional mesenchymal/stromal cell culture using the porous carriers in flow and plug-flow bioreactors.

### Example 3

### Superior Expansion and Growth of Hematopoietic Stem Cells from Unselected Mononuclear Cells

In order to test whether hematopoietic stem cells can be expanded from an unselected mononuclear cell fraction in the bioreactors, unselected mononuclear cells were seeded along with mesenchymal/stromal cells on carriers, and co-cultured in the flow bioreactor system. Expansion of hematopoietic stem cells (e.g. CD34+) from the unselected mononuclear cells was compared with that of cultures initiated with pre-selected, hematopoietic stem cells.

FIGS. 6a-6d show the surprisingly superior (greater than 10 times) fold expansion of hematopoietic stem cells (CD34+) cultured on carriers with human bone marrow stromal cells, especially during the first 14 days in culture, as compared with expansion from pre-selected CD34+ cells culture. FIGS. 7a and 7b, represent a FACS analysis of the hematopoietic stem cell population at 14 days culture. FIG. 7a-b demonstrated further evidence of the superiority of the expansion using unselected mononuclear cells the ability of mesenchymal cell culture to support the growth and expansion of hematopoietic stem cells (CD34+ and CD34+CD38− cells) better when unselected mononuclear rather than CD34+ selected cells were used to drive the process.

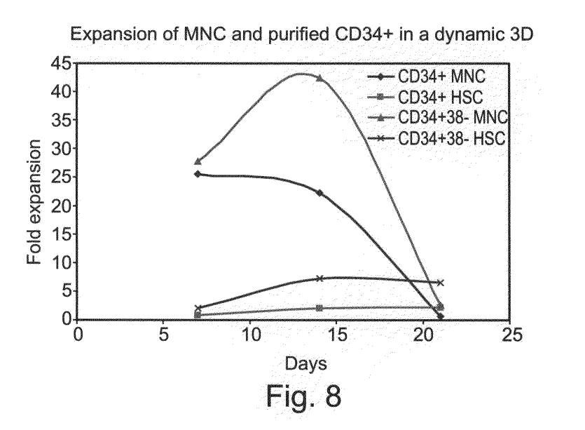

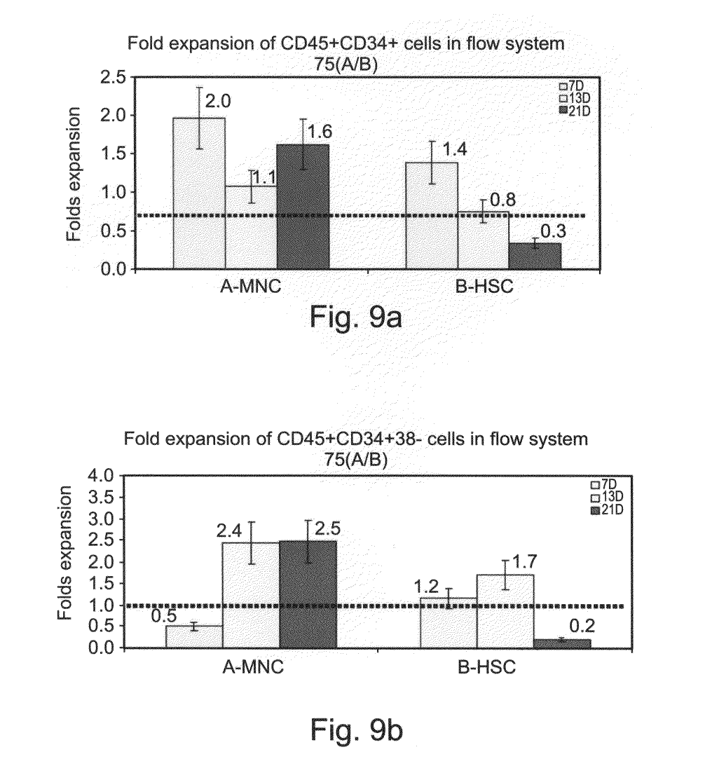

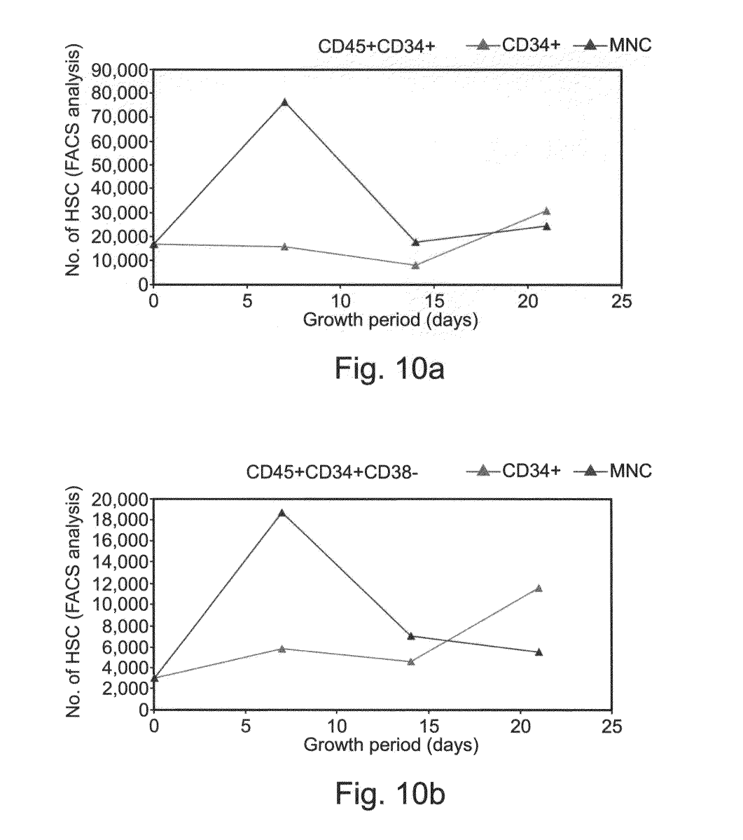

FIGS. 8 and 9a-9b provide yet further evidence for the strikingly efficient expansion of hematopoietic stem cell population from mononuclear cells co-cultured with bone marrow stromal cells, most prominent at 0-17 days, as compared with co-cultures initiated with pre-selected CD34+ cells. Co-culture of mononuclear cells with umbilical cord blood mesenchymal cells culture (FIGS. 10a-10b) indicated that the superior fold expansion of hematopoietic stem cells from mononuclear cells, especially at 0-15 days, can be achieved using a variety of mesenchymal/stromal cells cultured on carriers in bioreactors, according to the methods of the present invention.

Altogether these results demonstrate that co-culture, on three dimensional cultures, of mononuclear cells along with mesenchymal and/or stroma cells supports superior expansion of hematopoietic stem cells, without a requirement for added cytokines.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications cited herein are incorporated by reference in their entirety. Citation or identification of any reference in this application shall not be construed as an admission that such reference is available as prior art to the present invention.

### REFERENCES

1. Turhan, A. G., Humphries, R. K., Phillips, G. L., Eaves, A. C. & Eaves, C. J. N. Engl. J. Med. 320:1655, 1989.
2. Morrison, S. J., Uchida, N. & Weissman I. L. Ann. Rev. Cell. Dev. Biol. 11:35, 1995.
3. Ogawa, M. Blood 11:2844, 1993.
4. Lord B I, Testa N G, Hendry J H. Blood 45:65, 1995.
5. Trentin J J. in Gordon A (ed): Regulation of Hematopoiesis Vol I. New York, N.Y., Appleton-Century-Crafts, p 161, 1970.
6. Wolf N S. Clin. Hematol. 8:469, 1979.
7. Dorshkind K, Arm. Rev. Immunol. 8:11, 1990.
8. Allen T D, Dexter T M. Exp. Hematol. 12; 517, 1984.
9. Gupta P, McCarthy J B, Verfaillie C M. Blood 87:3229, 1996.
10. Liesveld J L, Winslow J M, Kempshi M C, Ryan D H, Brennan J K, Abboud C N. Exp. Hematol. 19:63, 1991.
11. Long M W, Briddel R, Walter A W, Bruno E, Hoffman R. J. Clin. Invest. 90:251, 1992.
12. Eaves C J, Cashman J D, Kay R J, Daugherty G J, Otsuka T, Gabury L A, Hogge D E, Landsdorp P M, Eaves A C, Humphries R K. Blood 78:110, 1991.
13. Moore K A, Pytowski B, Witte L, Hicklin D, Lemischka I. Proc. Nat. Acad. Sci. 94:4011, 1997.
14. Li L, Milner L A, Deng Y, Iwata M, Banta A, Graf L, Marcovina S, Friedman C, Trask B J, Hood L, Torok-Storb B. Immunity 8:43, 1998.
15. Allen T D, Dexter T M. Exp. Hematol. 12; 517, 1984.
16. Sutherland H, Landsdorp P M, Henkelman D, Eaves A C, Eaves C J. Proc. Nat. Acad. Sci. 87:3584, 1990
17. Breems D A, Blokland E A W, Nben S, Ploemacher R E. Leukemia 8:1095, 1994.
18. Verfaillie C, Blakolmer K, McGlare P. J. Exp. Med. 172:509, 1990.
19. Burroughs J, Gupta P, Blazar B, Verfaillie C. Exp. Hematol. 22:1095, 1994.
20. Roecklein B A, Torok-Storb B. Blood 85: 997, 1995.
21. Thiemann F T, Moore K A, Smogorzewska E M, Lemischka I R, Crooks G M. Exp. Hematol. 26:612, 1998.
22. Breems D A, Blokland E A W, Siebel K E, Mayen A E M, Engels L J A, Ploemacher R E. Blood 91:111, 1998.
23. Aiuti A, Friedrich C, Sieff C A, Gutierrez-Ramos J C. Exp. Hematol. 26:143, 1998.
24. Otsuka T, Satoh H, Ogo T, Bairy O, Gluck U, Zipori D, Nakano T, Okamura Y, Niho Y. Int. J. Cell Cloning 10:153, 1992.
25. Larochelle A, Vormoor J, Hahenberg H, Wang J C Y, Bhatia M, Lapidot T, Moritz T, Murdoch B, Xiao L X, Kato I, Willimas D A & Dick J E. Nat. Med. 2:1329, 1996.
26. Gan O I, MurdochB, Larochelle A, Dick J E. Blood 90:641, 1997.
27. Shultz L D, Schweitzer A, Christianson S W, Gott B, Shweitzer I B, Tennent B, McKenna S, Mobraaten L, Rajan T V, Greiner D L, Leiter E H. J. Immunol. 154:180, 1995.
28. Larochelle A, Vormoor J, Lapidot T, Sher G, Furukawa T, : I Q, Shultz L, Oliveri N F, Stamatoyannoppoulus G & Dick J E. Hum. Mol. Genet. 4:163, 1995.
29. Larochelle A, Vormoor J, Hahenberg H, Wang J C Y, Bhatia M, Lapidot T, Moritz T, Murdoch B, Xiao L X, Kato I, Willimas D A & Dick J E. Nat. Med. 2:1329, 1996.
30. Dick J E. Sem. Immunol. 8:197, 1996.
31. Bhatia M, Wang J C Y, Kapp U, Bonnet D, Dick J E. Proc. Natl. Acad. Sci. 94:5320, 1997.
32. Wang J C Y, Doedens M, Dick J E. Blood 89: 3919, 1997.
33. Peled A, Petit I, Kollet O, Magid M, Ponomaryov T, Nagler A, Ben-Hur H, Shultz L, Lider O, Alon R, Zipori D, Lapidot T. Science 283: 845-8. 1999
34. Bleul C C, Farzan M, Choe H, Parolin C, Clark-Lewis I, Legler D F, Loetscher M, Baggiolini M, Moser B. Nature 382:833, 1966.
35. Mizuno S, Wang J, Greenberger J, Glowacki J. Blood 88:189a (abs), 1996.
36. Rozenzweig M, Pykett M, Marks D F, Johnson R P. Gene Therapy 4:928, 1997.
37. Arakawa-Hoyt, Thiemann F T, Dao M A, Barsky L, Crooks G M, Nolta J A. Blood 92:581a, 1998 (Abst).
38. Moore K A, Pytowski B, Witte L, Hicklin D, Lemischka I. Proc. Nat. Acad. Sci. 94:4011, 1997.
39. Varnum-Finney B, Purton L E, Yu M, Brashem-Stein C, Flowers D, Staats S, Moore K A, Le Roux I, Mann R, Gray G, Artavanis-Tsakonas S, Bernstein I D. Blood 91:4084, 1998.
40. Verfaillie C M. Blood 79:2821, 1992.
41. Verfaillie C M. Blood 82:2045, 1993.
42. Bhatia R, McGlave P B, Miller J S, Wissink S, Lin W N, Verfaillie C M. Exp. Hematol. 25:980, 1997.
43. Breems D A, Blokland E A W, Ploemacher R E. Leukemia 11:142, 1997.
44. Herman P H, Ferrant A, De Bruyere M, Straetmans N. Leukemia 12:735, 1998.
45. Breems D A, Van Driel E M, Hawley R G, Siebel K E, Ploemacher R E. Leukemia 12:951, 1998.
46. Aiuti A, Ficara F, Dando J, ZE, Bordignon C. Blood 92:145a, 1998 (Abs).
47. Kusadasi N, van Soest P L, Ploemacher R E. Exp. Hematol. 25:699, 1998 (Abst).
48. Dorrell C, Gan O I, Pereira D S, Dick J E. Exp. Hematol. 25:688, 1998 (Abst).
49. Bhatia M, Bonnet D, Kapp U, Wang J C Y, Murdoch B, Dick J E. J. Exp. Med. 186:619, 1997.
50. Kollet O, Moore J, Fajerman I, Ben-Hur H, Hagay Z, Nagler A, Feldman M, Lapidot T. Blood 90:365a, 1997 (Abs).
51. Bertolini F, Battaglia M, Lanza A, Palermo B, Soligo D, Robustelli della Cuna G. Blood 90:365a, 1997 (Abs).
52. Luens K M, Travis M A, Chen B P, Hill B L, Scollay R, Murray U. Blood 91:1206, 1998.
53. Piacibello W, Sanavio F, Severino A, Dane A, Gammaitoni L, Fagioli F, Perissinotto E, Aglietta M. Blood. 93: 3736-49, 1999.
54. Kadouri A. Colloid and Surface B: Biointerface, 2:265, 1994.
55. Kadouri A, Kompier R, Honigwachs-Sha'anani J, Toledo J, Brosh N, Sternberg D, Levy A, Tzehoval E, Zipori D. Int. J. Cell Cloning 10:299, 1992.
56. Hansen M B, Nielsen S E, Berg K. J Immunol Methods 119:203, 1989.
57. Merchav S., Wagemaker G., Souza L. and Tatarsky I. Leukemia 5:344, 1991.
58. Kadouri A. Colloid and Surface B: Biointerface 2:265, 1994.
59. Kadouri A, Kompier R, Honigwachs-Sha'anani J, Toledo J, Brosh N, Sternberg D, Levy A, Tzehoval E, Zipori D. Int. J. Cell Cloning 10:299, 1992.
60. Zipori, D., Toledo, J. & von der Mark, K. Blood 66:447, 1985.
61. Otsuka T, Ogo T, Nakano t, Niiro H, Kuga S, Satoh H, Furukawa Y, Zipori D, Niho Y. Stem Cells 12:409, 1994.
62. Piacibello W, Sanavio F, Garetto L, Severino A, Bergandi D, Ferrario J, Fagioli F, Berger M, Aglietta M. Blood 89: 2644, 1997.
63. Carlo-Stella C, Mangoni L, Piovani G, Garau D, Almici C, Rizzoli V. Blood 83:1373, 1994.

What is claimed is:

1. A method of expanding/maintaining undifferentiated hematopoietic stem cells, the method comprising seeding unselected mononuclear cells into a stationary phase plug-flow bioreactor in which a three dimensional stromal cell culture has been pre-established on a substrate in the form of a sheet, wherein said substrate comprises a non-woven fibrous matrix forming a physiologically acceptable three-dimensional network of fibers, thereby expanding/maintaining undifferentiated hematopoietic stem cells.

2. The method of claim 1, wherein said mononuclear cells are cells isolated from a tissue selected from the group consisting of cord blood, peripheral blood, mobilized peripheral blood and bone-marrow.

3. The method of claim 1, wherein said unselected mononuclear cells comprise a cell population having a majority fraction of committed cells and a minority fraction of uncommitted, CD34+ cells.

4. The method of claim 3, wherein said minority fraction comprises from about 0.01% to about 30% of said cell population.

5. The method of claim 4, wherein said minority fraction comprises from about 0.01% to about 10% of said population.

6. The method of claim 5, wherein said minority fraction comprises from about 0.01% to about 5% of said population.

7. The method of claim 1, wherein stromal cells of said stromal cell culture are cells isolated from a source selected from the group consisting of bone cells, bone marrow cells, adipose tissue cells, placenta cells and umbilical cord cells.

8. The method of claim 1, wherein stromal cells of said stromal cell culture comprise mesenchymal stem cells.

9. The method of claim 1, wherein stromal cells of said stromal cell culture comprise adherent cells of a mesenchymal tissue.

10. The method of claim 1, wherein said mononuclear cells and stromal cells of said stromal cell culture share common HLA antigens.

11. The method of claim 1, wherein said mononuclear cells and stromal cells of said stromal cell culture are from a single individual.

12-14. (canceled)

15. The method of claim 1, wherein stromal cells of said stromal cell culture are grown to a density of at least 1×10⁶ cells per a cubic centimeter of said substrate.

16. The method of claim 1, wherein stromal cells of said stromal cell culture are grown to a density of at least 10⁷ cells per a cubic centimeter of said substrate.

17. The method of claim 1, wherein seeding said mononuclear cells into said stationary phase plug-flow bioreactor is effected while flow in said bioreactor is shut off for at least 2 hours following said seeding.

18. The method of claim 1, wherein said fibers form a pore volume as a percentage of total volume of from 40% to 95% and a pore size of from 10 microns to 100 microns.

19. The method of claim 1, wherein said matrix is made of fiber selected from the group consisting of flat, non-round, and hollow fibers and mixtures thereof, said fibers being of from 0.5 microns to 50 microns in diameter or width.

20. The method of claim 1, wherein said matrix is composed of ribbon formed fibers having a width of from 2 microns to 20 microns.

21. The method of claim 1, wherein said matrix having a pore volume as a percentage of total volume of from 60% to 95%.

22. The method of claim 1, wherein the matrix has a height of 50-1000 μm.

23. The method of claim 1, wherein the material of the matrix is selected from the group consisting of polyesters, polyalkylenes, polyfluorochloroethylenes, polyvinyl chloride, polystyrene, polysulfones, cellulose acetate, glass fibers, and inert metal fibers.

24. The method of claim 1, wherein the matrix is in a shape selected from the group consisting of squares, rings, discs, and cruciforms.

25. The method of claim 1, wherein the matrix is in the form of a disc.

26. The method of claim 1, wherein the matrix is coated with poly-D-lysine.

27. The method of claim 1, further comprising the step of isolating said mononuclear cells.

28. A method of transplanting undifferentiated hematopoietic stem cells into a recipient, the method comprising the steps of:
(a) expanding/maintaining the undifferentiated hematopoietic stem cells according to the method of claim 1; and
(b) transplanting said undifferentiated hematopoietic stem cells resulting from step (a) in the recipient.

29-54. (canceled)
